(19) United States
(12) Patent Application Publication
(10) Pub. No.: US 2010/0028925 A1
(43) Pub. Date: Feb. 4, 2010

(54) METHOD AND DEVICE FOR THE ANALYSIS OF ENZYME ACTIVITY IN FLUIDS

(76) Inventor: Richard M. Rocco, Alameda, CA (US)

Correspondence Address: SCHNECK & SCHNECK, P.O. BOX 2-E, SAN JOSE, CA, 95109-0005, US

(21) Appl. No.: 12/510,465

(22) Filed: Jul. 28, 2009

Related U.S. Application Data
(60) Provisional application No. 61/084,475, filed on Jul. 29, 2008.

(51) Int. Cl. C12Q 1/42

(52) U.S. Cl. 435/21

(57) ABSTRACT

An assay in which alkaline phosphatase is detected on one of a number of membranes held on a holder. A liquid sample and a substrate are combined on the membrane. If the enzyme is present, the substrate is cleaved. The products of this cleavage are optically detectable.

### CROSS-REFERENCE TO RELATED APPLICATION

This application claims priority from provisional application Ser. No. 61/084,475 filed Jul. 29, 2008.

### TECHNICAL FIELD

The present invention relates to analysis of enzyme activity and more specifically to optical analysis of enzyme activity.

### BACKGROUND

The enzyme alkaline phosphatase (ALP, EC 3.1.3.1) is widely distributed in human and animal tissues. H. D. Kay in 1930 developed a method of analysis for ALP in biological fluids. Researchers soon established the utility of measuring elevated levels of ALP in human serum as a marker for a wide variety of bone and liver diseases. Today, ALP assays are a routine part of clinical laboratory medicine. Kay and Graham in 1933 expanded the use of the ALP assay into the food safety industry. They demonstrated that the ALP activity of raw bovine milk decreased in concentration in proportion to the temperature and time used to pasteurize the milk. The level of ALP activity in a processed fluid dairy product could now be used to determine if the product had been properly pasteurized or if the product had been contaminated post-pasteurization with raw milk. Proper pasteurization had been shown to reduce the levels of ALP in raw milk from over 100 000 mU/L to below 500 mU/L. Deviations in pasteurization temperature of as little as 0.1° C. resulted in product with over 500 mU/L. Incursion of raw milk into a finished dairy product (e.g., through a leaky valve) would also result in elevated levels of ALP in the finished dairy product. The ALP assay for dairy product safety and pasteurization monitoring soon became an established industry and regulatory assay. By the late 1940's the US FDA and the European International Dairy Federation (IDF) established legal limits for the sale of fluid dairy products based on ALP levels. This legal limit is still enforced to this day and has expanded to all industrialized countries.

Many methods for the measurement of ALP in fluid dairy products have been described over the years. The two most important considerations for all ALP methods used in this product safety application are (a) speed of analysis and (b) limit of detection. Dairy products have a limited shelf life post-pasteurization. Busy pasteurization production facilities cannot afford to delay shipment of product while waiting for assay results. The early ALP methods, for example, took up to 3 hours to complete. The second challenge for this assay is the detection limit. For over fifty years the legal limit for fluid dairy products has been set at 500 mU/L. ALP methods had to distinguish between a dairy product with 250 mU/L and 500 mU/L. By way of contrast ALP methods used in the clinical medical field for disease detection in humans only had to distinguish between a 250 000 mU/L and a 500 000 mU/L. The orders of magnitude lower levels required for pasteurization monitoring in fluid dairy products increased the technical challenges for the ALP assays used in the dairy industry.

In 1935 Kay and Graham introduced the first practical method for ALP in dairy products. The assay measured the hydrolysis of the substrate phenyl phosphate by ALP. Liberated phenol was measured with the Folin-Ciocalteu reagent. The intensity of the blue color was read in a calorimeter and the assay took 24 hours to complete. A shorter version was also described in which the developed blue color was read visually after a 15 minute incubation time. The limit of detection for this assay was at the required legal limit of 500 mU/L. A breakthrough in testing methods occurred in 1938 when Harry Scharer introduced his improved version of the Kay and Graham method. He used Gibbs reagent (2,6-dichloroquinone-4-chloroimide) to measure the liberated phenol instead of the Folin-Ciocalteu reagent. This allowed the assay time to be shortened to 10 minutes. The blue color could be read with a calorimeter after extraction of the chromophore with butanol or visually. Scharer obtained U.S. Pat. No. 2,359,052 on this method on Sep. 26, 1944. Despite the need to extract the blue phenol color with butanol prior to the reading, the Scharer method went on to become an industry standard for over 50 years.

Kosikowsky improved on the Scharer method by eliminating the need for butanol extraction of the phenol color complex. He incubated the milk sample and substrate mixture in a cellulose dialysis sack. The liberated phenol diffused through the dialysis sack into a buffer. Following addition of Gibb's reagent the blue color was read free from the turbidity and interference of the milk sample. The assay took one hour and Kosikowsky was issued U.S. Pat. No. 3,293,147 on Dec. 20, 1966.

In 1949 Aschaffenburg and Mullen introduced the use of p-nitrophenol phosphate as a substrate for the milk ALP assay. The liberated p-nitrophenol in alkaline solution was yellow and required no additional color developing reagents. A visual assay was possible after 15 minutes incubation and a quantitative calorimetric assay required 2 hours. Like all previous methods the assay could detect down to but not below the legal limit of about 500 mU/L.

Babson and Greeley introduced both a visual and a quantitative calorimetric method for ALP in milk with use of the self-indicating substrate phenolphththalein monophosphate in 1967. In this assay the liberated phenolphthalein developed a red color in alkaline buffer and did not require any additional color developing reagents. A visual reading could be taken in 15 minutes. The substrate phenolphthalein monophosphate for use with all types of ALP assays was covered in the U.S. Pat. No. 3,002,893 issued Oct. 3, 1961.

The first major improvement in ALP methods for dairy products occurred in 1990 with the introduction of a fluorometric substrate for the milk ALP assay. In this assay 2′[2-benzothiazoyl]-6′-hydroxybenzothiazole phosphate is cleaved to release the highly fluorescent 2′[2-benzothiazoyl]-6′-hydroxybenzothiazole product. Because of the increased sensitivity obtained with the use of fluorescence the assay could be run in 3 minutes and for the first time reproducible results down to about 30 mU/L of ALP activity in milk were now possible. FDA and IDF approvals and widespread adoption of this assay soon followed its introduction. For the first time since the introduction in the late 1930's of the ALP assay for milk safety and quality control, the FDA in 2000 lowered the legal limit. The regulatory standard is now 350 mU/L. All previous calorimetric methods in use in the dairy industry and by regulatory agencies had a detection limit of above this limit which limited their applicability.

In 2006 Salter and Fitchen described the use of a chemiluminescent substrate for the measurement of ALP in dairy products. Chemiluminescent 1,2-dioxetane substrates are widely used in biotechnology. With chemiluminescence they were able to match the lowered detection limits of the fluorometric assay.

Both the fluorometric and the chemiluminescent assays have replaced the older calorimetric assays that are unable to detect ALP at the current regulatory limits of below 500 mU/L. Both of these newer methods however still suffer from a number of technical disadvantages. First, they do not provide a visual interpretation of the results. All previous calorimetric methods used in the dairy industry were based on visual inspection of the sample after color development. This helped provide a further check on the quality control of dairy products. Second, multiple different samples run at the same time is difficult to achieve. Both methods require numerous pipeting steps with use of glass test tubes. The test tubes are then read one at a time for up to three minutes each in a dedicated fluorometer or luminometer. Severe price and budget constrictions in the dairy industry has limited the introduction of robotic directed automated analyzers. In fact, cost per test is one of the major considerations in this segment of the food industry.

The purpose of disclosed embodiments is to provide a low-cost means for the measurement of ALP in dairy products. The embodiments allow for multiple samples to be assayed at the same time and provides a visual confirmation of the results along with quantitative reporting of the results of the enzyme activity in mU/L. This invention provides a low-cost platform that also meets the requirements for a detection system that can measure ALP activity well below 35 mU/L or up to ten times below the regulatory requirement.

### SUMMARY OF THE INVENTION

A method of detecting alkaline phosphatase in a liquid sample, in which a selected volume of liquid sample is added into one of a plurality of membranes. Each membrane is secured onto a holder that is substantially free from an optical background, such that the holder will not interfere with optical signal even at the bottom range of detection. A fluorescent or chemiluminescent substrate is then added onto each membrane, where it combines with the samples. If alkaline phosphatase is present in the liquid sample, the enzyme will cleave the substrate, and the result of the cleavage will be optically detectable. This may be done visually or using an optical imaging system. The use of a calibration membrane with a known amount of sample may allow for a simpler quantification of the amount of alkaline phosphatase in the sample.

### BRIEF DESCRIPTION OF THE DRAWINGS

FIG. 1 is a top view of a disposable device for the assays.

FIG. 2 is a top view of a disposable device showing one elevated sample.

FIG. 3 is a top view of a disposable device showing one elevated sample and having a centrally placed calibrator.

### DETAILED DESCRIPTION

The disclosed embodiments include a disposable platform for the assays and a digital imaging reader.

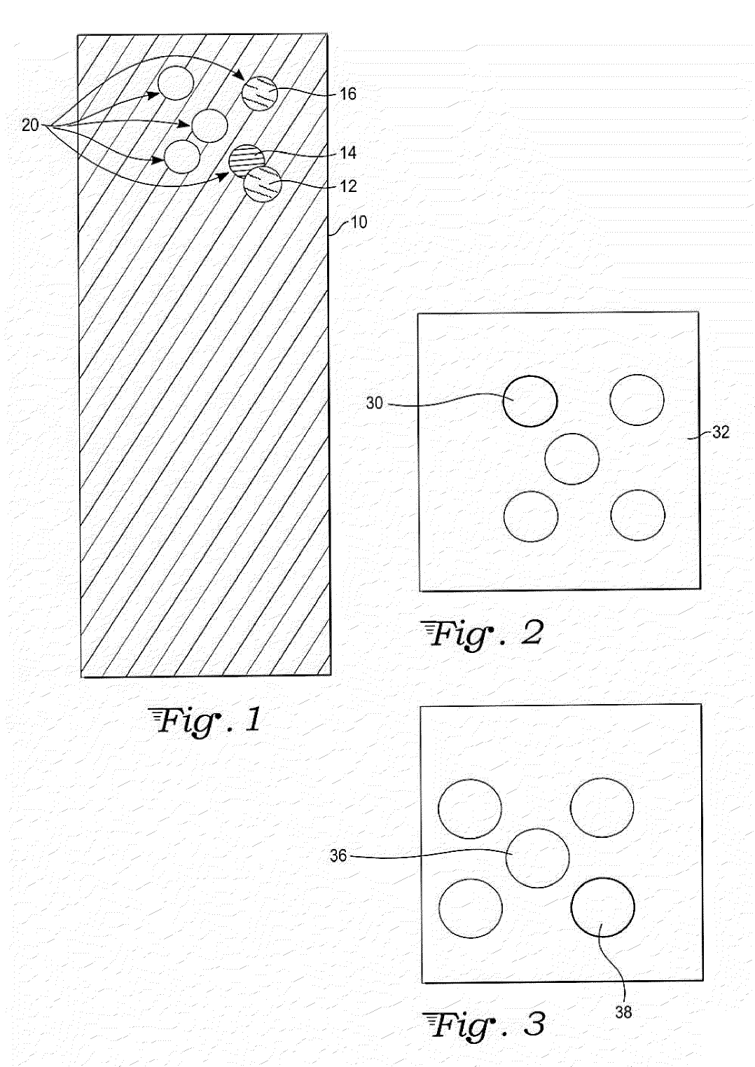

An illustrated disposable device shown in FIG. 1 includes a 2.5 cm wide by 9 cm long and 0.8 mm thick plastic sheet 10 having up to five holes punched in it. Each hole is 5 mm in diameter. The plastic sheet is opaque gray or black and has no fluorescent or chemiluminescent background (i.e. is substantially free from optical background). One side of the plastic sheet is lined with a clear transparent polyethylene tape 14 where the holes 20 have been punched through the sticky side of the tape is exposed. Into the holes are placed 5 mm circles of a membrane 12 chosen for use in this assay. The membrane dots are held in place by the sticky side of the tape exposed in the bottom of the hole. Holes in the plastic holder and the membranes are cut with a standard hole punch. Both Whatman 3MM chromatography and Whatman GF/F glass fiber membranes have been used. Both type membranes are opaque white with no background fluorescence or chemiluminescence. Each membrane in its respective well forms a location for one assay of a milk sample for ALP activity. The hole in the plastic sheet forms a well in which the membrane is confined. The milk sample and substrate, when added to the membrane, provides a uniform mixing compartment and a concentration chamber for the enzyme product being formed and measured in the assay. In essence this is a quantitative enzyme assay format in which the entire assay is performed in a small (less than 5 mm diameter) dot format. Quantitation of enzyme activity is through standard digital image analysis image capture and software analysis rather than through conventional absorbance, fluorescence or chemiluminescence intensity captured through conventional calorimeters, spectrophotometers, or luminometers.

A typical assay consists of adding a defined sample volume of the unknown milk sample to the dry membrane, typically 5-10 uL. The milk sample is absorbed into the membrane and remains confined within the diameter of the membrane within the well. Substrate is then added to each well. Either fluorescent or chemiluminescent substrate at 20-30 uL can be used. The liquid volume of substrate is absorbed and confined within the membrane and is mixed within the membrane with the milk sample through simple diffusion. The reaction of ALP with the substrate is then allowed to proceed for a selected period of time, typically three minutes. Multiple plastic holders may be incubated on a standard flat plate incubator. Either the fluorescent or chemiluminescent product formed by the action of ALP in the milk within the membrane dot is also confined and concentrated within this geometric space. Typical membranes used are 0.4 to 1.0 mm in thickness and from less than 1 to over 10 mm in diameter.

### Reader:

The reader used to measure the ALP activity in each membrane dot is determined by the type of substrate used. For either chemluminescent or flurometric substrate a digital image is captured of the dots on the plastic holder. Digital imaging devices for capturing images of fluorescent or chemiluminescent spots or immuno dot-blots in biotechnology are readily available. Some low cost units utilize a readily available hand-held digital CCD camera. In all cases the intensity of the staining of the spots are analyzed with standard imaging software. For chemiluminescent imaging the plastic holder is placed within a light tight box and the photons of light emitted from the dot are captured with a CCD camera or photomultiplier tube (PMT) detector. For the capture of fluorescence images the holders are placed on the surface of a standard fluorescent light box and the images captured with a CCD, PMT or CMOS camera. These fluorescent light boxes are readily available and contain filter elements to match the required fluorophore. Fluorescence and chemiluminescence image capture boxes are currently used to measure the staining intensity of stained biomaterials in an electrophoresis gel or on membrane sheets which have blotted the proteins of interest from the electrophoresis gels followed by antibody-enzyme linked immuno-staining of the blotted proteins bands.

### EXAMPLE

Commercial samples of store-bought pasteurized whole milk were diluted with unpasteurized whole milk to provide samples with various concentrations of ALP from 10 to 1000 mU/L. Up to five different samples at 10 uL each were added to individual dots on a holder. Fluorometric substrate was then added to each dot at 20 uL. The dots were cut from standard Whatman 3MM chromatography membranes. The holders were allowed to incubate for 3 minutes followed by imaging of the dots in a home-made fluorescence light box with a mounted digital camera. Digital images were downloaded from the camera into Apple iPhoto software and each dot analyzed for intensity with standard software. The image analysis software is freely downloadable from the National Institutes of Health (NIH) and is called ImageJ software. FIG. 1 shows a filter dot holder. FIG. 2 is a typical image of a fluorescent assay captured with a standard CCD camera held above a light box illuminator. The one sample 30 on the holder 32 with above 350 mU/L of ALP and is above the regulatory limit which requires that the product not be sold for human consumption is easily identified as the brightest spot. FIG. 3 shows another holder with five samples. In this case the center dot 36 is a milk calibrator at 100 mU/L against which the values for the remaining four samples are calculated in mU/L of ALP activity. Spot 38 is well above the calibrated standard 36.

The above illustrative examples could be characterized in a number of different ways including:
(1) An assay format for the measurement of enzyme activity in which the entire assay is analyzed in a membrane dot.
(2) The design of the holder in which the dot membrane is isolated from other dots with no physical contact between dots. This provides a unique location for each unknown sample being assayed.
(3) The measurement of enzyme activity by digital imaging of the product formed per unit time within each dot. The analysis of the digital image for dot intensity and enzyme activity calculation is performed with any standard imaging software.
(4) An enzyme assay format in which the results for the unknown samples are visually presented. The visual presentation of the results is a unique feature of this assay format.
(5) An enzyme assay format which can be used for calorimetric, fluorometric or chemiluminescent assays.
(6) A rapid enzyme assay for ALP in dairy products in which multiple samples can be run; the required lower detection limit is achieved and which also provides a visual confirmation of the results.
(7) The membrane that forms the base in which the assay is performed may be made of cellulose; nitrocellulose, PVP; glass fiber or any other biocompatible woven or non-woven material.
(8) Dot sizes can vary from less than 1 mm to over 10 mm and is limited only by the field of view of the optics employed to capture the image.
(9) Image capture and analysis may be based on colored images (as for calorimetric or fluorometric assays) or black and white chemiluminescent assays which capture images in a single wavelength of photons.
(10) By increasing the number of membrane dots multiple enzyme assays can be run on a single holder. The number of dots is limited only by the field of view of the optical image capture system.
(11) Different enzymes may be analyzed on the same holder with the same sample. A single sample can be added to multiple wells and then each well in turn is charged with a different reagent or set of reagents for each different enzyme assay being run on that same sample.
(12) An ALP assay for fluid dairy products that can be used to check for completeness of pasteurization or the incursion of raw milk into a finished dairy product.
(13) An ALP assay for solid dairy products (cheese, yogurt etc) following a standard extract of the sample as currently used in the dairy industry when testing for ALP in these type of products. For example, when testing for high levels of ALP in cheese to determine if the non-aged cheese was made from raw or pasteurized milk.
(14) An enzyme assay format that can be used for a wide variety of assays in the food, beverage, pharmaceutical, environmental and health related fields when an enzyme assay is utilized for quality inspection and safety control purposes.
(15) An enzyme assay format for ALP in dairy products in which one of the dot membranes is impregnated with a milk calibrator and dried. Murthy and Peeler described the preparation of these calibrators or controls and demonstrated their long-term stability at room temperature (12). In their application 0.25 inch circles of the filter paper disks were dropped into the calorimetric substrate as a substitute for the preparation of fresh control material.
(16) An ALP assay for measuring the completeness of pasteurization in milk and milk products made from milk obtained from a wide variety species of animals, example cow, sheep, goat, camel and other mammals. This includes human milk. The ALP assay is used to monitor for completeness of pasteurization of human milk stored in human milk banks.
(17) A unique aspect to this device is that milk samples during production can be applied to the dot membranes and allowed to dry. For experimental, quality control or regulatory purposes the samples can be easily transported to a central laboratory for analysis at a latter date. Fluid dairy products have a typical refrigerator shelf life of 10 days. After this time retrospective analysis of ALP activity in the milk sample for quality control trace back or regulatory purposes are impossible because the milk has been consumed or destroyed. Samples collected during processing at the dairy facility could be stored and analyzed at a latter date. Membranes similar to those described in this application have been used to collect human blood samples for storage and analysis at a latter date of a wide variety of markers in neonatal genetic screening procedures.

The prior description of ALP measurement in dairy products is but one example of the presently developed technology. There are a number of further different ways of categorizing the present invention.

(1) For a fluorescent, calorimetric or luminescent assay, obtain a polymer substrate that has a plurality of holes extending through the substrate. The hole size can vary from less than 1 mm to over 10 mm and is dependent on the optical detection system. The substrate must have low luminescent or fluorescent background in the wavelengths used for assay detection. Clear biocompatible tape is applied to one side such that the sticky side of the tape is exposed through the hole in the substrate. Membrane circles or dots are cut from the membranes and placed into the holes. In the example give the substrate had five holes each 5 mm in diameter. These numbers and dimensions are exemplary and higher densities are anticipated.
(2) An assay format in which on a single substrate a plurality of membranes are secured. Each membrane has sufficient size to allow a sample to be placed onto the membrane and reagents to be placed on the membrane, to provide a calorimetric, fluorescent or luminescent result. The membrane thickness is chosen to allow the sample and reagent mixture to remain enclosed or trapped within the membrane. Assay examples in this format include enzyme assays, endpoint assays and spot calorimetric assays.
(3) The membranes may be pre-impregnated with the reagents, which could be dried onto the membrane. This provides a very simple assay in which the sample could simply be placed onto the membrane on a substrate and the membranes on the substrate analyzed after a suitable interval. Alternatively, the samples could be placed on the membrane and the membrane stored at room temperature or at refrigerated temperatures according the requirements for the reagents. This would allow the samples to tested at a latter time.
(4) The present format assays were read with a simple reader constructed from readily available parts and assembled in the following manner. The intention of the basic design is to provide a digital image of the membrane spots, importation of the image into a standard computer and analyzed for calorimetric, fluorometric or luminescent intensity using readily available image analysis software.

### Colorimetric Assays

These assays include simple color spot tests and enzymatic assays in which the enzyme develops a color spot in direct proportion to the enzyme concentration.

(a) Membrane spots may be imaged using a simple hand-held digital camera such as a Pentax Optio 6.0 megapixel digital camera readily available in retail outlets.

(b) Images are imported into any standard computer photo analysis software such as Apple iPhoto.

(c) The images can then be analyzed for quantitative intensity and calculation of sample results using any standard image analysis software. One example is the freely downloadable image analysis software from the NIH called ImageJ analysis software. This will provide qualitative and quantitative results for each of the membrane spots.

### Fluorometric Assays

(a) Light boxes are readily available from a wide range of laboratory supply houses such as VWR or Fisher Scientific. Alternatively a light box may be constructed from readily available supplies. A standard light box consists of a light source such as a fluorescent lamp or LCD lamp mounted inside an enclosure. An excitation filter is placed above the light, the wavelength of the filter chosen for the fluorophore being utilized in the assay. The membrane substrate with added sample and reagents are placed onto the top of the filter followed by placement of an emission filter chosen for the fluorophore being used in the assay.

(b) Image analysis follows the image capture as above.

(c) Image analysis software follows the software utilization as above.

### Luminescent Assays

(a) For these assays a simple light tight box with attached CCD camera is utilized to collect images of the developed membrane spots.

(b) Image analysis follows the image capture as above.
(5) Assays

Spot analysis using calorimetric analysis of a wide variety of assays in environmental, food safety, clinical and pharmaceutical analysis have been described for many years (for example, Spot Test Analysis. Clinical, Environmental, Forensic, and Geochemical Applications, E. Jungreis, 1985). In these assays the color developed on the membrane is read visually in a semi-quantitatve manner. The present invention extends this form of assays in the following different embodiments.

(a) Use of the sample and reagent in a membrane dot or circle format.
This provides a concentration effect and an increase in the possible detection limit or limit of quantitation.

(b) Application of readily available imaging technology to replace the human eye for analysis.

(c) Application of standard software image analysis software to provide quantitative analysis of results.

(d) Extension of the spot analysis format to flurometric and luminescent analysis of the developed images.

What is claimed is:

1. A method to assay alkaline phosphatase content in a liquid sample, the method comprising:
adding a selected volume of liquid sample each onto one of similarly sized membranes, each membrane secured onto a holder, said holder substantially free from a fluorescent or luminescent background;
adding a fluorescent or chemiluminescent substrate onto each sample, said substrate producing an optical signal if cleaved by alkaline phosphatase; and
optically detecting the optical signal from each membrane.

2. The method of claim 1, wherein the liquid sample is milk.

3. The method of claim 1, wherein one of the membranes is a calibration membrane having a known amount of alkaline phosphatase.

4. The method of claim 1, wherein optically detecting the signal includes visually inspecting the membranes.

5. The method of claim 1, wherein optically detecting the signal includes capturing multiple membranes in a single image.

6. The method of claim 1, wherein the time between adding the substrate and detecting is three minutes or less.
